(19) United States
(12) Patent Application Publication
(10) Pub. No.: US 2005/0031550 A1
(43) Pub. Date: Feb. 10, 2005

(54) Antigingival/anticalculus oral rinse composition and method of use

(76) Inventor: Joseph B. Busch, Edison, NJ (US)

Correspondence Address: CLIFFORD G. FRAYNE, Suite 7A, 136 Drum Point Road, Brick, NJ, 08723, US

(21) Appl. No.: 10/636,147

(22) Filed: Aug. 7, 2003

(51) Int. Cl. A61K 7/16; A61C 5/00

(52) U.S. Cl. 424/49; 433/217.1

(57) ABSTRACT

There is disclosed an antigingival/anticalculus oral rinse composition comprised of an aqueous solution of vinegar, sodium bicarbonate and sodium chloride, together with other adjuvant compounds wherein vinegar is present in an amount greater than sodium bicarbonate and sodium chloride and sodium bicarbonate is present in an amount greater than sodium chloride.

### RELATED APPLICATIONS

Applicant claims the benefit of provisional application Ser. No. 60/401,217, filed Aug. 8, 2002.

### BACKGROUND OF THE INVENTION

1. Field of the Invention

The present invention relates to oral hygiene, and more particularly, to an improved antigingival/anticalculus oral composition and method of use.

2. Description of the Prior Art

Dental plaque is a mucous film about the teeth harboring diverse forms of bacteria and is a precursor to the formation of calculus or tartar which may form on any part of a tooth surface but is particularly sited about the gingival margins. Such forms of bacterial include pathogenic microorganisms associated with periodontal disease, dental infections and caries.

Present protocols directed to attacking dental plaque include medical devices, such as brushing, flossing, and the like which are more effective on contacting surfaces whereas surfaces which cannot be conveniently contacted, i.e. areas between teeth are more effectively attacked by chemical agents comprised of an active antiplaque organic, inorganic compounds and mixtures thereof as well as dental mechanical devices, such as scrappers.

Organic compounds include cetyl pyridinium chloride (CPC), cholorohexidiene Triclosan, etc., such as discussed in U.S. Pat. No. 5,043,154 to Gaffer et al, which are not effective with water soluable polyphosphates salts. Additional art, such as U.S. Pat. Nos. 4,894,220 and 5,047,635 to Nabi et al, U.S. Pat. No. 5,037,637 to Gaffer et al., U.S. Pat. No. 5,032,385 to Reid et al. are further disclosures directed to antiplaque/anticalculus compositions.

While much effort has been directed to more efficacious formulations to remedy such problems, there remains the need for an antiplaque/anticalculus composition of greater effectiveness at reduced costs.

### OBJECTS OF THE PRESENT INVENTION

An object of the present invention is to provide an improved antiplaque/anticalculus formulation of greater efficacy.

Still another object of the present invention is to provide an improved antiplaque/anticalculus formulation in a protocol of improved effectiveness.

Yet another object of the present invention is to provide an improved antiplaque/anticalculus formulation of improved effectiveness against gingival fluids.

A still further object of the present invention is to provide an improved antiplaque/anticalculus formulation effective to remediate existing conditions of gingival attack.

### SUMMARY OF THE PRESENT INVENTION

These and other objects of the present invention are achieved by an antiplaque/anticalculus oral rinse composition comprised of an aqueous solution of vinegar, sodium bicarbonate and sodium chloride, together with other adjuvant compounds wherein vinegar is present in an amount greater than sodium bicarbonate and sodium chloride and sodium bicarbonate is present in an amount greater than sodium chloride as more fully hereinafter disclosed.

### DETAILED DESCRIPTION OF THE INVENTION

In accordance with a preferred embodiment of the present invention, it has been found that a synergistic oral antiplaque/anticalculus result is achieved by an aqueous solution comprising vinegar, sodium bicarbonate and sodium chloride wherein vinegar is present in an amount proportionately greater than sodium bicarbonate and sodium chloride and sodium bicarbonate is present in an amount proportionately greater than sodium chloride. The composition includes other adjuvant compounds to provide a physiologically acceptable product, etc.

In the form of a generic oral rinse composition, the following Table I lists the compositions and the range thereof:

TABLE I

| INGREDIENT | % RANGE |
|---|---|
| H₂O | 30-70 |
| Vinegar (Acetum) | 10-20 |
| Sodium Bicarbonate | 5-10 |
| Sodium Chloride | 1-5 |
| Adjuvant Compounds | Remainder |

In the form of a preferred embodiment an oral rinse composition, the following Table II lists the compositions and the range thereof:

TABLE II

| INGREDIENT | % RANGE |
|---|---|
| H₂O | 30-70 |
| Vinegar (Acetum) | 10-20 |
| Glycerin | 10-20 |
| Sodium Bicarbonate | 5-10 |
| Sodium Chloride | 1-5 |
| PEG-40 Hydrogenated Castor Oil | 1-5 |
| Polysorbate-20 | 1-5 |
| Flavor (Aroma)* | 0.1-1 |
| Sodium Citrate | 0.1-1 |
| Sodium Benzoate | 0.01-1 |
| Sodium Saccharin | 0.01-1 |
| Sodium Laureth Sulfate | 0.01-1 |
| Methylparabenzene | 0.5-1 |
| Green 3 (Cl 42053) | 0.01-1 |
| Yellow 5 (Cl 1 0140) | 0.01-1 |

*Flavor Lipment 244-497 AFF@0.5%

In use, of from 1 to 3 ounces of the composition of the present invention is introduced into the oral cavity and caused to be vigorously swished within the oral cavity for at least about 30 seconds before expectorating to attack the bacteria present in the oral cavity as well as to break down calculus formations. Accordingly, such procedure also improves gum adhesion to the teeth.

In a procedure of use, it is preferable that a user first floss and then brush the teeth with an appropriate toothpaste, it being understood that the flossing could follow brushing. Thereafter, the user follows the hereinabove described rinsing process. An additional brushing following the rinse process facilitates plaque removal as well as bacterial debris loosened by the rinse process and suspended therein.

The following is an example of an exemplar formulation of the oral rinse composition of the present invention:

### EXAMPLE I

| COMPONENT | % BY WEIGHT |
|---|---|
| Water (Aqua) | 59.9240 |
| Vinegar (Acetum) | 14.0000 |
| Glycerin | 12.0000 |
| Sodium Bicarbonate | 6.0000 |
| Sodium Chloride | 2.8000 |
| PEG-40 Hydrogenated Castor Oil | 2.0000 |
| Polysorbate-20 | 2.0000 |
| Flavor (Aroma) | 0.5000 |
| Sodium Citrate | 0.2500 |
| Sodium Benzoate | 0.0200 |
| Sodium Saccharin | 0.0600 |
| Sodium Laureth Sulfate | 0.0625 |
| Methylparaben | 0.3000 |
| Green 3 (CI 42053) | 0.0260 |
| Yellow 5 (Cl 19140) | 0.0575 |
| | 100.0000 |

While the present invention in a preferred form is an oral rinse composition, it will be understood by one skilled in the art that the basic componets, i.e. vinegar, sodium bicarbonate and sodium chloride may be incorporated into a toothpaste, which when used prior to the rinse process would be still more effective in attacking plaque and calculus in abating the effect of gingivitis. Thus, of from about 10 to 20% by volume of the basic componets would be incorporated into a toothpaste formulation.

Use of a toothpaste formulation following the oral rinse process would provide a still more further effective protocol in providing improved oral hygiene results. It is understood that the term vinegar is used in the context of commercial available acetic acid of specific concentrations, and that the term is intended to cover dilute acetic acid solutions.

While the present invention has been described with respect to the exemplary embodiments thereof, it will be recognized by those of ordinary skill in the art that many modifications or changes can be achieved without departing from the spirit and scope of the invention. Therefore it is manifestly intended that the invention be limited only by the scope of the claims and the equivalence thereof.

1. An improved method for attacking in an oral cavity bacterial precursors for calculus on tooth formation which comprises:
introducing into said oral cavity an aqueous solution of vinegar, sodium bicarbonate and sodium chloride, together with adjuvant compounds.
causing said aqueous solution to be coursed within said oral cavity about and between teeth in said oral cavity for a time sufficient to permit destruction of said bacteria in said oral cavity; and
removing a resulting aqueous solution from said oral cavity.

2. The improved method as defined in claim 1 wherein venegar is present in an amount greater than sodium bicarbonate and sodium chloride and sodium bicarbonate is present in an amount greater than sodium chloride.

3. The method as defined in claim 2 wherein vinegar is present in an amount of from 10 to 20 percent by weight, sodium carbonate is present in an amount of from 5 to 10 percent by weight and sodium chloride is present in an amount of from 1 to 5 percent by weight.

4. An oral rinse composition comprised of the following ingredients in the cited percent by weight:

| INGREDIENTS | % BY WEIGHT |
|---|---|
| H₂O | 30-70 |
| Vinegar | 10-20 |
| Sodium Bicarbonate | 5-10 |
| Sodium Chloride | 1-5 |
| Adjuvant Compounds | Remainder |

5. An oral rinse composition comprised of the following ingredients in the cited percent by weight.

| INGREDIENT | % RANGE |
|---|---|
| H₂O | 30-70 |
| Vinegar (Acetum) | 10-20 |
| Glycerin | 10-20 |
| Sodium Bicarbonate | 5-10 |
| Sodium Chloride | 1-5 |
| PEG-40 Hydrogenated Castor Oil | 1-5 |
| Polysorbate-20 | 1-5 |
| Flavor (Aroma)* | 0.1-1 |
| Sodium Citrate | 0.1-1 |
| Sodium Benzoate | 0.01-1 |
| Sodium Saccharin | 0.01-1 |
| Sodium Laureth Sulfate | 0.01-1 |
| Methylparabenzene | 0.5-1 |
| Green 3 (Cl 42053) | 0.01-1 |
| Yellow 5 (Cl 1 0140) | 0.01-1 |

*Flavor Lipment 244-497 AFF@0.5%
